(19) United States
(12) Patent Application Publication
(10) Pub. No.: US 2012/0061257 A1
(43) Pub. Date: Mar. 15, 2012

(54) CONCENTRIC BIPOLAR ELECTROCHEMICAL IMPEDANCE SPECTROSCOPY TO ASSESS VASCULAR OXIDATIVE STRESS

(75) Inventors: FEI YU, Los Angeles, CA (US); TZUNG K. HSIAI, Santa Monica, CA (US); EUN S. KIM, Rancho Palos Verdes, CA (US)

(73) Assignee: UNIVERSITY OF SOUTHERN CALIFORNIA, Los Angeles, CA (US)

(21) Appl. No.: 13/232,828

(22) Filed: Sep. 14, 2011

Related U.S. Application Data
(60) Provisional application No. 61/403,317, filed on Sep. 14, 2010.

(51) Int. Cl. G01N 33/50; G01N 27/30

(52) U.S. Cl. 205/792; 204/406; 204/403.01

(57) ABSTRACT

Concentric bipolar electrode sensors and assemblies provide for and/or facilitate detection and diagnosis of the non-obstructive and pro-inflammatory atherosclerotic lesions in human arteries during catheterization using electrochemical impedance spectroscopy (EIS). Fabrication techniques for concentric bipolar electrode sensors are also described.

### CROSS-REFERENCE TO RELATED APPLICATION

Application claims priority to and benefit of U.S. Provisional Patent Application Ser. No. 61/403,317 filed Sep. 14, 2010, Attorney Docket No. 028080-0603, and entitled “Concentric Bipolar Electrochemical Impedance Spectroscopy to Assess Vascular Oxidative Stress,” the entire content of which is incorporated herein by reference.

### STATEMENT REGARDING FEDERALLY SPONSORED RESEARCH

This invention was made with Government support under Contract Nos. NHLBI 083015 and NHBLI 068689, awarded by the National Institutes of Health. The Government has certain rights in the invention.

### BACKGROUND

Detection of atherosclerotic lesions prone to rupture is of utmost importance in the management of patients with acute coronary syndromes or stroke. Despite the advent of computed tomographic (CT) angiography, high resolution MRI, intravascular ultrasound (IVUS), near-infrared fluorescence (NIRF), time-resolved laser-induced fluorescence spectroscopy, and other techniques, predicting metabolically active atherosclerotic lesions has remained an unmet clinical need. Mechanically unstable atherosclerotic plaque is often characterized by a thin-cap fibrous atheroma (<65 μm) and a metabolically active lipid core. Rupture of these plaques is clinically manifested as acute coronary syndromes or stroke. Emerging imaging modalities such as an integrated intravascular ultrasound (IVUS) and optical coherence tomography (OCT) system have enabled the colocalization of thin-cap fibroatheroma with intimal hyperplasia and calcification. Assessing metabolic states in the inflammatory, albeit non-obtrusive, lesions has remained a diagnostic challenge.

### SUMMARY

Concentric bipolar electrodes sensors and assemblies are used for electrochemical impedance spectroscopy (EIS) to measure impedance of biological tissue and bio-materials.

An aspect of the present disclosure is directed to a system for electrochemical impedance spectroscopy (EIS). The system can include a concentric bipolar electrode sensor assembly including an outer electrode and a center electrode disposed within the outer electrode. The concentric bipolar electrode assembly can be configured to supply an excitation voltage across the outer and center electrodes. The system can include a memory, storage device, or memory unit that is configured to receive data from the concentric bipolar electrode assembly. A processor can be included that can be connected to the memory. Programming can be included for execution by the processor, and stored in the memory or storage device. Execution of the programming by the processor configures the system to perform functions, including functions to measure impedance across the outer and center electrodes for EIS measurements of biological tissue or material adjacent to the sensor assembly.

A further aspect of the present disclosure is directed to an article of manufacture including a non-transitory machine-readable storage medium; and executable program instructions embodied in the machine readable storage medium that when executed by a processor of a programmable computing device configures the programmable computing device to: from electrical signals received from a bipolar concentric electrode sensor, measure impedance of biological tissue or material; and provide an output signal indicative of the measure impedance to a display device.

While certain embodiments are depicted in the drawings, one skilled in the art will appreciate that the embodiments depicted are illustrative and that variations of those shown, as well as other embodiments described herein, may be envisioned and practiced within the scope of the present disclosure.

### BRIEF DESCRIPTION OF THE DRAWINGS

The drawing figures depict one or more implementations in accord with the present teachings, by way of example only, not by way of limitation. They do not set forth all embodiments. Other embodiments may be used in addition or instead. Details that may be apparent or unnecessary may be omitted to save space or for more effective illustration. Conversely, some embodiments may be practiced without all of the details that are disclosed. When the same numeral appears in different drawings, it refers to the same or like components or steps. The drawings are not necessarily to scale, emphasis instead being placed on the principles of the disclosure. In the drawings:

FIG. 1 depicts a concentric bipolar electrode in accordance with the present disclosure.

FIGS. 2A-2B depict an exampled of a process of constructing a concentric bipolar electrode sensor assembly according to the present disclosure.

FIG. 3 depicts another example of a process of constructing a concentric bipolar electrode sensor assembly according to the present disclosure.

FIG. 4 depicts a set of diagrams of equivalent circuits and impedance characteristics graphs according to the present disclosure.

FIG. 5 depicts a set of photographs of fatty streak tissue with accompanying endoluminal electrochemical impedance spectroscopic measurements according to the present disclosure.

FIG. 6 depicts a set of photographs of oxLDL-rich fibrous atheroma tissue with accompanying endoluminal electrochemical impedance spectroscopic measurements according to the present disclosure.

FIG. 7 depicts a set of photographs of oxLDL-absent fibrous atheroma tissue with accompanying endoluminal electrochemical impedance spectroscopic measurements according to the present disclosure.

FIG. 8 depicts a set of photographs of calcification with accompanying endoluminal electrochemical impedance spectroscopic measurements according to the present disclosure.

FIG. 9 depicts a set of graphs showing the sensitivity and specificity of endoluminal electrochemical impedance spectroscopic measurements for an implemented embodiment of a concentric bipolar electrode sensor according to the present disclosure.

FIG. 10 shows Table 1, which lists a classification scheme for atherosclerotic lesion stages, and Table 2, which shows a comparison of simulated circuit parameters for the equivalent circuits shown in FIG. 4.

While certain embodiments are depicted in the drawings, one skilled in the art will appreciate that the embodiments depicted are illustrative and that variations of those shown, as well as other embodiments described herein, may be envisioned and practiced within the scope of the present disclosure.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, and/or circuitry have been described at a relatively high-level, without detail, in order to avoid unnecessarily obscuring aspects of the present teachings.

Systems and methods of the present disclosure provide for and/or facilitate detection and diagnosis of the non-obstructive and pro-inflammatory atherosclerotic lesions in human arteries during catheterization by use of concentric bipolar electrodes for electrochemical impedance spectroscopy (EIS). Biological tissues store charges, and electric impedance (Z) develops as a function of frequency in response to the applied alternating current (AC), and accordingly, atherosclerotic lesions can display distinct electrochemical properties. Active lipids and macrophages cause distinct electrochemical properties in the vessel wall that can be measured by electrochemical impedance spectroscopy (EIS). Distinct electrochemical properties of oxidized low density lipoprotein (oxLDL) and foam cell infiltrated in the subendothelial layer at lesion sites can be measured in terms of the electrochemical impedance spectroscopy (EIS) using concentric bi-polar electrodes as described herein. Concentric bipolar microelectrodes can accordingly be used to measure electrochemical impedance in regions of pro-inflammatory states with high spatial resolution. Distinct from linear four point electrode arrays, methods and systems according to the present disclosure employ concentric bipolar electrodes, allowing for reproducible assessment for vascular regions harboring vascular oxidative stress in terms of oxLDL and foam cells. Concentric electrodes can provide constant and symmetric displacement between working and counter electrodes. Moreover, concentric configuration may allow for EIS measurement independent of the surrounding solutions or blood and the orientation of the tissues. Because of the micro-scale of the concentric electrodes, the impedance measurement is mainly sensitive to the electrochemical properties of the tissue at close proximity, thus during in vivo investigation the impedance measurements may be largely independent of lumen diameters, blood volumes, and flow rates when the contact is made between microelectrodes and endoluminal surface. In implemented embodiments, specimens that harbored oxidative stress were found to generate distinctly higher EIS values compared to the healthy tissues over a range of frequency from 10 KHz to 100 kHz; other frequency ranges may of course be utilized.

EIS sensors according to the present disclosure can be incorporated onto a steerable catheter accompanied with intravascular ultrasound (IVUS) to scan the circumferential profile of the atherosclerotic vasculature. EIS measurement can be performed at multiple sites for a single lesion to generate a contour map containing both topographical and electrochemical information. In addition, EIS measurements can be potentially incorporated with intracardiac echocardiogram, optical coherence tomography (OCT), and/or micro-thermal sensors to further enhance the sensitivity and specificity for the assessment of pro-inflammatory states or unstable plaque.

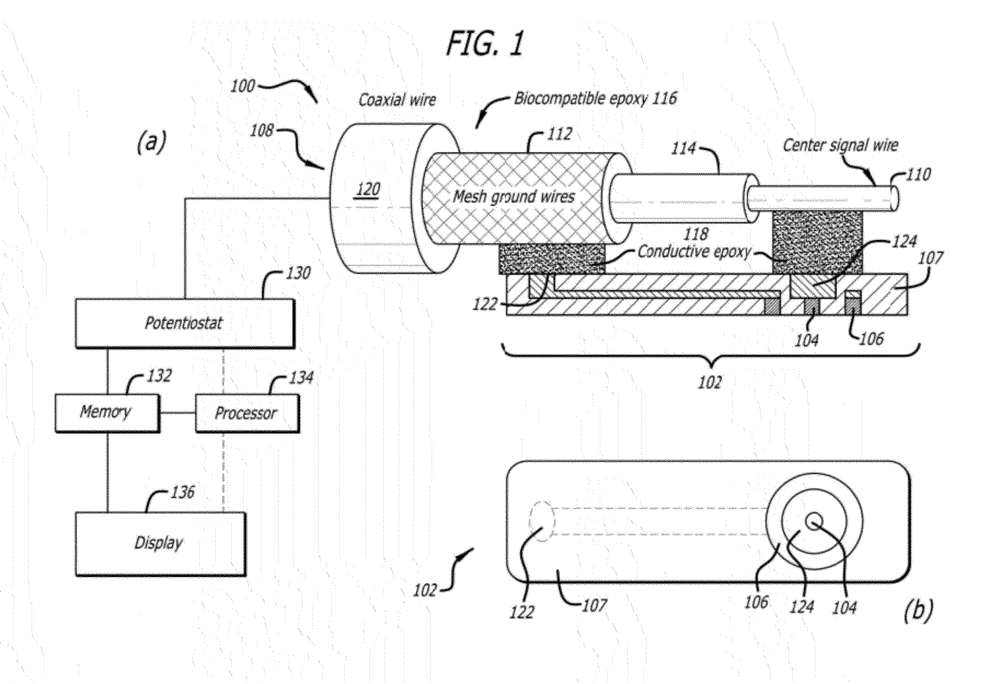

FIG. 1 depicts a concentric bipolar electrode sensor assembly 100 in accordance with the present disclosure. View (a) is a side cross section view of sensor 100, while view (b) is a bottom view of sensor 102. As shown, sensor assembly 100 includes a sensor 102 including a pair of concentric electrodes including an inner or center electrode 104 and an outer electrode 106, which are configured concentrically in a body or layer 107 of nonconductive material, e.g., parylene or the like. Sensor 102 is connected to a coaxial wire 108, which includes a central signal wire 110 and a mesh ground wire portion 112 configured in a coaxial and concentric configuration separated by an insulating layer 114. Coaxial wire 108 can include an outer protective sheath or layer 120 as shown. Conductive epoxy 118 can be used to attach sensor 102 to the coaxial wire 108 at an outer electrode binding site 112 and a center electrode binding site 124. A biocompatible epoxy 116 may be used to seal or enclose the end of assembly, as shown, e.g., for delivery to a lumen of a patient's vasculature. Coaxial wire 108 may provide electrical connection to various components or systems for applying voltage with a desired waveform to electrodes 104 and 106, e.g., a suitable waveform generator capable of supplying AC voltage of a desired frequency, e.g., between 500 kHz and 10 Hz inclusively. Coaxial wire 108 may provide electrical connection to various components or systems for measuring impedance across the electrodes 104 and 106, e.g., a potentiostat, for taking EIS measurements of the impedance of tissue and/or material in proximity to the electrodes, e.g., non-obstructive and pro-inflammatory atherosclerotic lesions in human arteries. The impedance measurements can be used to detect the presence or absence of types of tissue or disease states, e.g., by correlating to or matching known measured impedances for such tissues and materials. Any suitable materials may be used for the electrodes and conductive wire. Examples include, but are not limited to, steel, platinum, gold, silver, copper, alloys of such, and the like.

As further shown in FIG. 1, system 100 can include a device to measure impedance, or impedance measuring device 130, such as potentiostat. Any suitable imaging system may be utilized for the impedance measuring device 130. The impedance measuring device 130 can provide impedance data to a memory unit 132 and processor 134. The memory unit 132 and/or processor 134 may be connected to a display 136 as shown. Any suitable memory unit, e.g., amount of RAM and/or ROM, may be used. Further, any suitable processor 134 may be used. For example, the processor 134 may be a general central processing unit (CPU) or a graphics-specialized graphics processing unit (GPU). Of course, the architecture is flexible, and the processor 134 may optionally be directly coupled to imaging system and/or display 136. Any suitable display, of any suitable size and/or type, may be used for display 136. The processor 134 and/or memory unit 132 may provide output signals indicative of measured impedance to the display 136. The processor 134 may include or run suitable software (programming, or computer-readable instructions resident in a computer-readable storage medium) for measuring impedance. Examples of suitable imaging software include but are not limited to MATLAB, e.g., MATLAB Release 2011b, as made commercially available by the MathWorks, and Gamry Echem Analyst, e.g., EIS300 Electrochemical Impedance Spectroscopy Software, as made commercially available by Gamry Instruments. Such software, when appropriately modified or programmed to implement embodiments of the present disclosure.

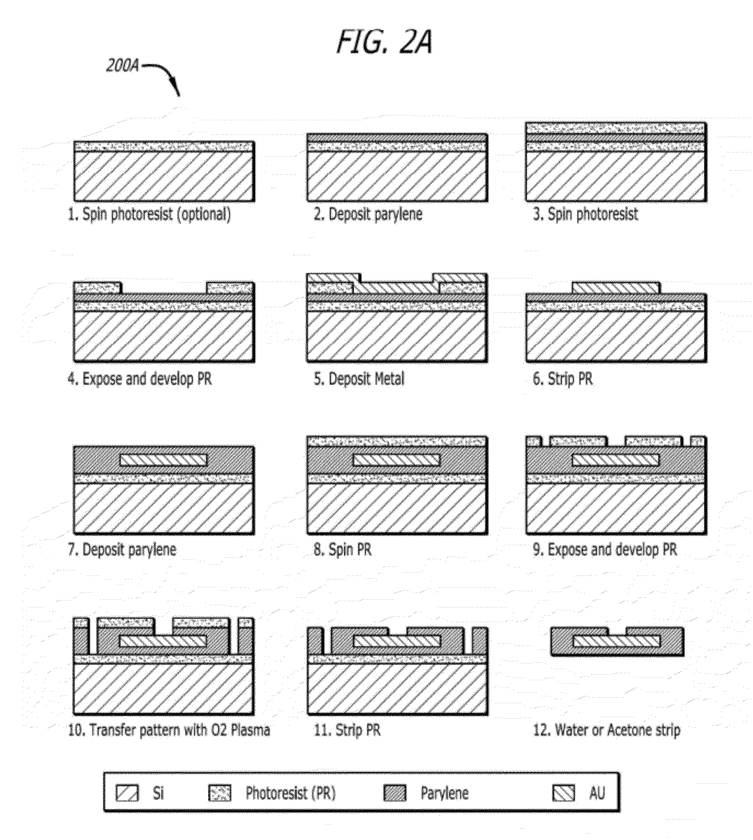

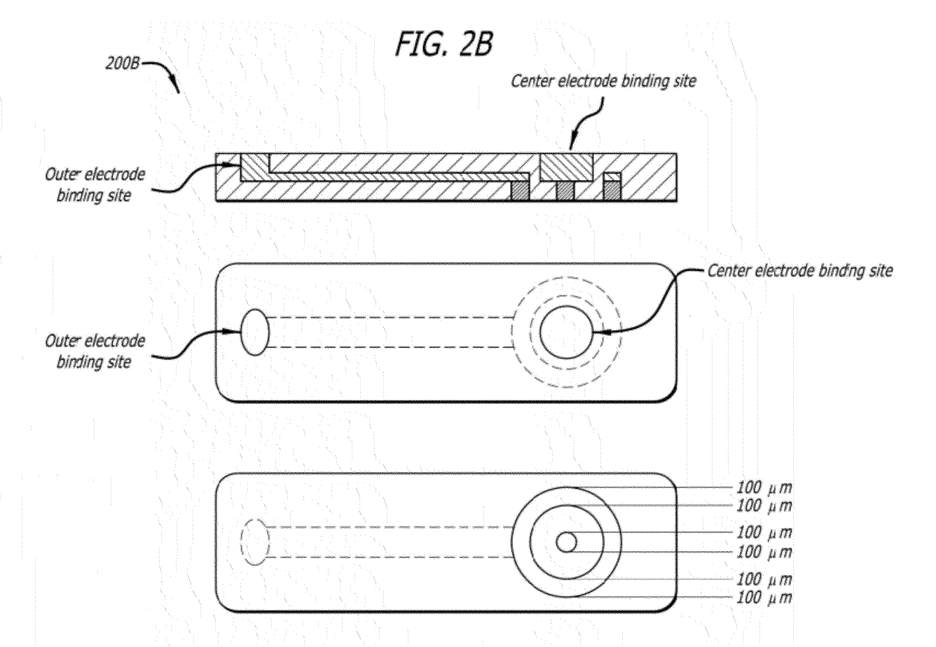

Details for a method of fabricating 200 an exemplary concentric bipolar electrode assembly, in the form of a MEMS based sensor, are shown in FIGS. 2A-2B. As shown at step 1 of FIG. 2A, spin photoresist can be applied to a substrate. As shown as step 2, parylene can be deposited on the substrate. A layer of photoresist may be applied to the parylene, as shown at step 3. The photoresist can be exposed and developed into a desired pattern for metal deposition and then stripped, as shown at steps 4-6. A layer of parylene can be deposited and covered with photoresist, as shown at steps 7-8. The photoresist can be patterned, exposed, and developed, as shown at step 9. The resulting pattern can be transferred with O2 plasma and stripped, as shown at steps 10-11. The assembly can then be stripped with water or acetone, as shown at step 12 in FIG. 2A. FIG. 2B shows the resulting sensor ready for binding to a coaxial wire, e.g., coaxial wire 108 in FIG. 1.

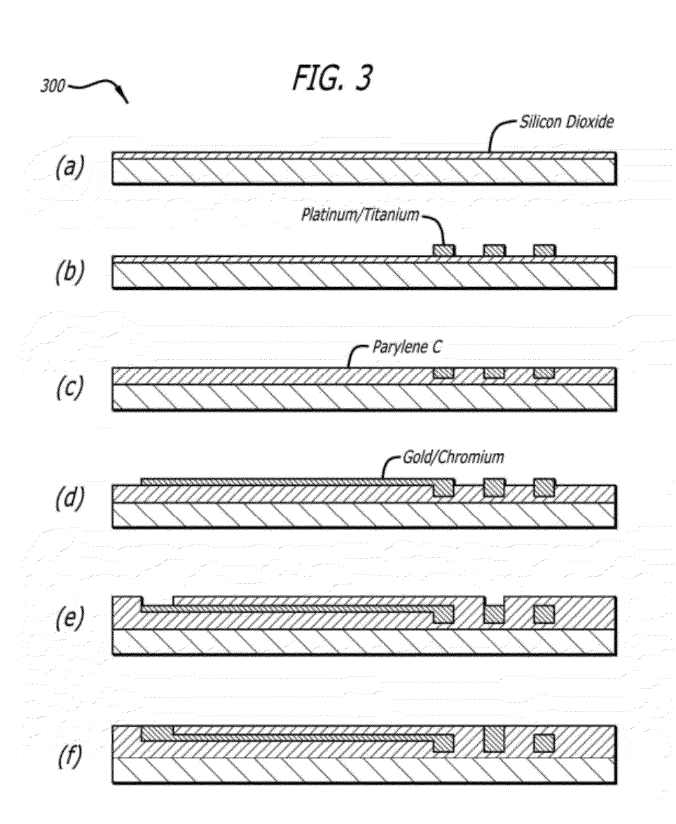

FIG. 3 shows another example of a fabrication process 300 for making a concentric bipolar electrode sensor according to the present disclosure. Step (a) shows thermal growth of SiO2 and deposition of sacrificial Si layer (e.g., 1 μm in thickness). Step (b) illustrates deposition and patterning of Ti/Pt layers (e.g., 2 μm in thickness) for the concentric electrode. Step (c) shows deposition and patterning of parylene C (e.g., 2 μm in thickness). Step (d) depicts deposition and patterning of a metal layer of Cr/Au for electrodecontact (e.g., 2 μm in thickness). At step (e), deposition and patterning of a thick layer of parylene C are shown (e.g., 10 μm in thickness) to form the device structure. Step (f) shows deposition and patterning of Cr/Au for electrode leads (e.g., 2 μm in thickness). Further in FIG. 3, step (g) illustrates etching the underneath Si sacrificial layer to lift the sensor, along with top and bottom view of the sensor. Step (h) illustrates packaging of the resulting MEMS EIS concentric bipolar electrode sensor. The sensor was connected to the electrical coaxial wire with conductive epoxy and covered with biocompatible epoxy to prevent electrical current leakage. The distance between the binding site of center and outer electrodes is about 1 cm to avoid possible short-circuitry.

### Exemplary Implemented Embodiments

Exemplary embodiments were tested and demonstrated the ability to characterize metabolically active lesions via EIS measurements in explants of human aorta. Equivalent circuit models were developed to assess electric circuit parameters in the context of simulating endoluminal EIS measurements. EIS measurements performed on 15 coronary, carotid, and femoral arteries at various Stary stages of atherosclerotic lesions revealed distinct electrochemical impedance spectroscopic signals. Endoluminal impedance was significantly higher in the active lipid-rich lesions as validated by positive anti-oxLDL staining. To corroborate the specificity of EIS measurements, significant differences in frequency-dependent impedance signals were demonstrated among fatty streaks (Stary Type II lesions), thin fibrous cap oxLDL-rich (Type III or IV), oxLDL absent fibroatheroma (Type V), and calcified lesions (type VII).

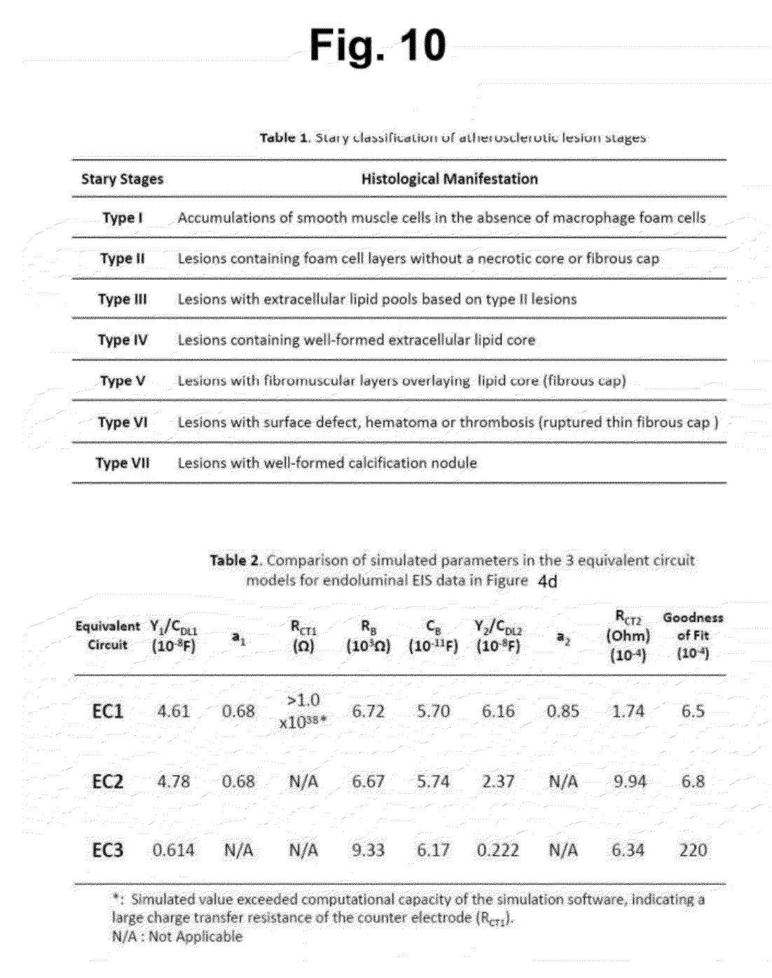

Regarding material and methods, fresh-frozen human artery specimens were obtained from National Disease Research Interchange (NDRI) in accordance with the University of Southern California Institutional Review Board guideline. A total of 15 human coronary, carotid and femoral arterial segments from nine (9) donors were analyzed for endoluminal EIS measurements. The arterial samples were immersed in phosphate buffered saline (PBS) solution (commercially available from Invitrogen, CA, USA), and sectioned longitudinally to unfold the endoluminal sides. The gross pathology of individual specimens revealed various degrees of atherosclerosis as classified by Stary stages from type I to VII, as shown in Table 1 of FIG. 10. A total of 147 points of interest with gross lesion-free condition or various types of atherosclerotic lesions were selected for endoluminal EIS measurements.

EIS measurements were conducted using the concentric bipolar microelectrodes with a flat tip profile (commercially available from FHC Co., ME, USA). Briefly, the concentric bipolar microelectrode was mounted vertically on a micro-manipulator (commercially available from World Precision Instruments, FL, USA), and made in contact with tested tissue at selected measuring point. An Ag/AgCl electrode (commercially available from World Precision Instruments, FL, USA) was used as the reference electrode. Frequency-dependent impedance was measured from 100 Hz to 300 kHz (commercially available from Gamry Series G 300 potentiostat, PA, USA). The magnitudes and phases of the EIS measurements were recorded at 20 data points per frequency decade, and the measured impedance spectrums were analyzed (commercially available from Gamry Echem Analyst software, PA).

To simulate equivalent circuit model for the concentric bipolar electrode-endoluminal tissue interface for an implemented embodiment, three models describing both working and counter electrode interface as well as the tissue impedance were constructed.

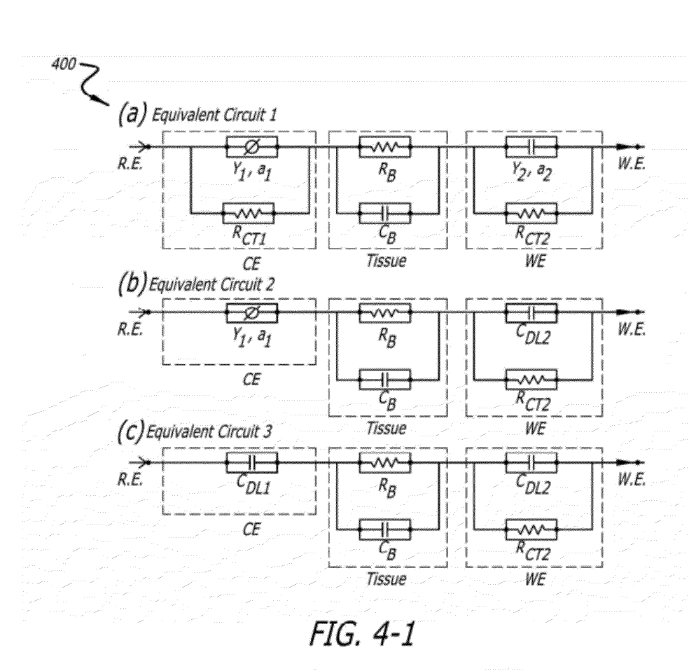

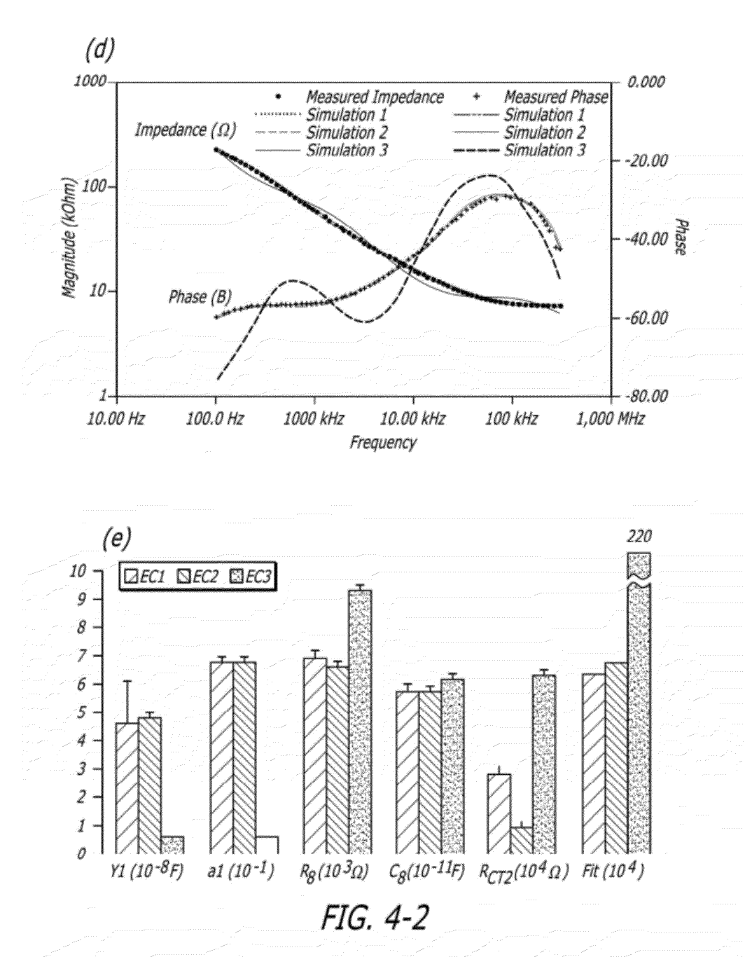

FIG. 4 depicts a set 400 of diagrams of equivalent circuits and impedance characteristics graphs according to the present disclosure. Equivalent circuit models shown in FIG. 4 were used to simulate electrochemical impedance spectrum measured between the concentric bipolar electrodes.

As shown in FIG. 4a, Equivalent Circuit model 1 (EC1) was composed of six electric elements. Both counter electrode (CE) and working electrode (WE) were denoted as a constant phase elements in parallel with a charge transfer resistance. The impedance of constant phase element Z_{CPE} can be expressed as:

$${Z}_{CPE} = \frac{1}{{Y ( jω )}^{a}} \qquad ( EQ . 1 )$$

where Y represents the capacitance and a is the empirical constant describing the surface property of an electrode. When a=1, the CPE operates as an ideal capacitor. In most cases, a fell between 0 and 1 (0<a<1), where Z_{CPE} indicates the non-ideal behavior of electrode-tissue interface capacitance. The charge transfer resistance, R_{CT}, was seen as being predominantly dependent on the chemical and physical properties of electrolyte solution and electrode material. Each blood vessel was considered to harbor both resistive (R_{B}) and capacitive (C_{B}) properties; thus, both were seen as contributing to the overall tissue impedance. The R_{B} and C_{B} values were mainly dependent on the composition and structure of the tissue, particularly its water, lipid, ion and charged molecule content. With the assumption of an extremely large charge transfer resistance (R_{CT1}) at large-area counter electrode and an ideal double layer capacitance (C_{DL2}) replacing the CPE at working electrode, Equivalent Circuit model 2 (EC2) was implemented to avoid potential over-fitting to simplify the simulation (FIG. 4b). In such context, the total number of parameters was reduced to six. In addition, by assuming that CPE at counter electrode also acts as an ideal double layer capacitance (C_{DL1}), Equivalent Circuit model 3 (EC3) further reduced total parameters to five (FIG. 4c). The best-fitting model parameters (R_{B}, C_{B}, Y, a, R_{CT} and C_{DL}) for the three equivalent circuits were simulated, using a simplex algorithm in the Gamry Echem Analyst software. The difference between the simulated and experimental impedance was represented as a single “Goodness of Fit” value, equivalent to the square of the overall percentage error. Using this approach, a comparison was made of the agreement between the simulated and experimental spectra, and to verify the equivalent circuits as the best suited model for concentric bipolar endoluminal EIS applications.

Regarding histology and immunohistochemistry, human specimens were sectioned and immersed in 4% paraformaldehyde for paraffin fixation immediately following the impedance measurements. Multiple slides with thickness of 5 μm were cut for histological evaluation. Standard hematoxylin and eosin (H&E) staining was performed to visualize initima, media, smooth muscle cells, adventitia, and foam cells. The metabolic states of atherosclerotic lesions were assessed by anti-oxLDL antibody (mAb4E6) to active lipids, Oil-red-O to lipid content, and von Kossa to calcification. All histological sections were visualized under Olympus IX70 microscopes (made commercially available by Olympus, Japan) and captured with a CCD digital camera (such as a ProGres® C3, made commercially available by Jenoptic, Germany). Metabolically active conditions of the fibroatheromas were classified by the Stary stages in terms of intimal hyperplasia, thin-cap atheroma, active lipids, and calcification.

For statistical analysis, atherosclerotic lesions were categorized into five types (lesion free/fatty streak/thin cap oxLDL-rich atheroma/oxLDL-absent fibroatheroma/calcified lesions) based on histological evidence. Due to variations in specimen size, thickness, and possible changes in electrode surface chemistry after multiple applications, inter-specimen variations in baseline EIS measurements could develop. To standardize comparisons, all of the parameter values obtained from simulation were normalized to the respective mean parameter values obtained from the lesion-free sites of the same specimens. Next, one-way analysis of variance (ANOVA) and two-tailed T-test were used for multi-group comparison and comparison between lesion and lesion free groups, respectively. P values <0.05 were considered statistically significant.

As for results, an optimal equivalent circuit to simulate endoluminal EIS measurements were constructed as shown for Equivalent Circuits 1 and 2 (EC1 and EC2). Both Equivalent Circuits 1 and 2 (EC1 and EC2) predicted frequency-dependent changes in impedance (Ω) and phase, and were in agreement with the endoluminal EIS measurements in the human carotid arteries accompanied by approximately 2.5% error, as shown in FIG. 4d).

Equivalent Circuit 3 (EC3) predicted frequency-dependent changes in impedance accompanied by approximately 14.8% error and by a significant deviation in the phase values (θ). The individual circuit parameters were further compared among the three equivalent circuits, as shown in FIG. 4e and Table 2 of FIG. 10. The comparison revealed that R_{B}, C_{B} and Goodness of Fit values were nearly identical between equivalent circuits 1 and 2. Given that the estimated value of charge transfer resistance (R_{CT1}) in the counter electrode of EC1 exceeded the computational limit (10³⁸Ω) of the software (Gamry Echem Analyst), the high R_{CT1} was removed from EC2. However, the assumption that CPE in the counter electrode functioned as a double layer capacitor (C_{DL1}) resulted in a decrease in Goodness of Fit and a deviation in phase (θ) values in EC3. In this context, EC2 provided the optimal model to simulate EIS results in the concentric bipolar electrode-endoluminal tissue interface, and established the basis for the ensuing analysis of endoluminal EIS measurements.

Endoluminal EIS measurements were compared between fatty streak-rich and fatty streak absent sites, followed by immunohistochemistry analysis for anti-oxLDL and Oil-red-O staining.

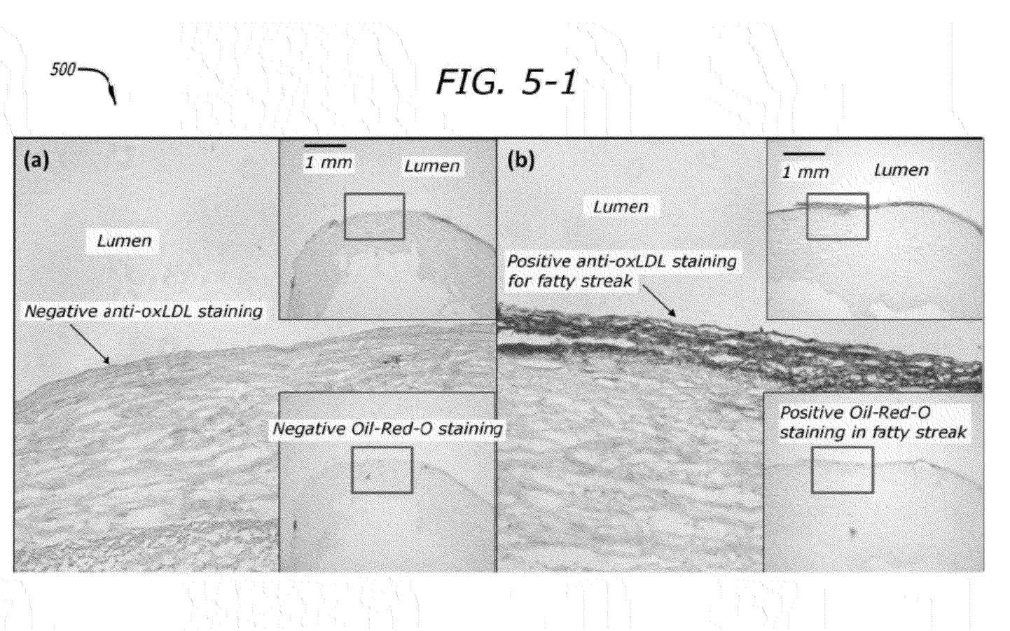

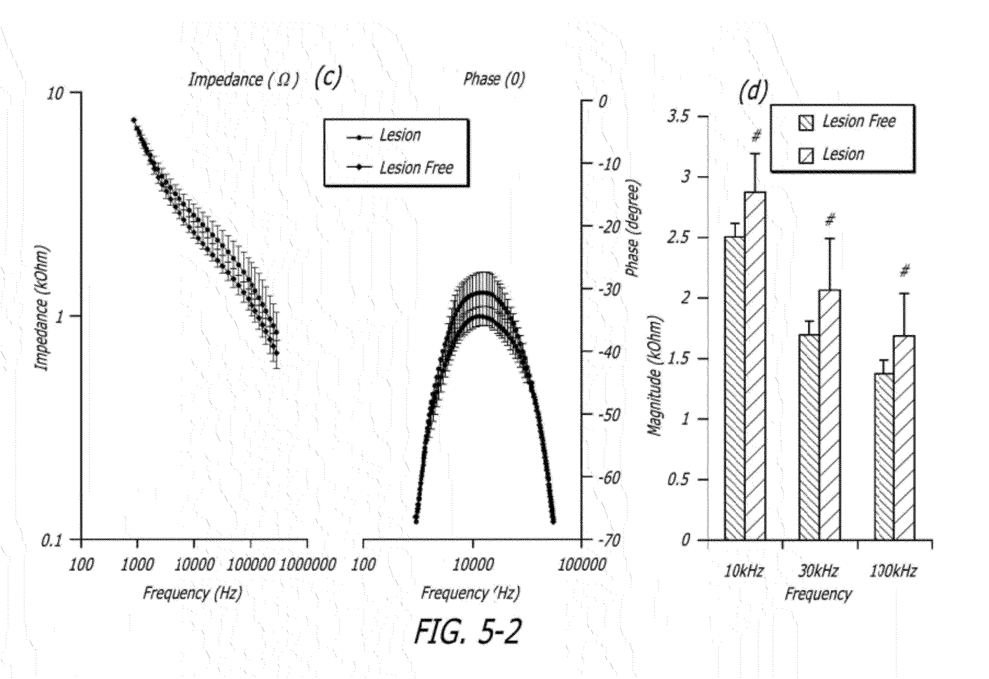

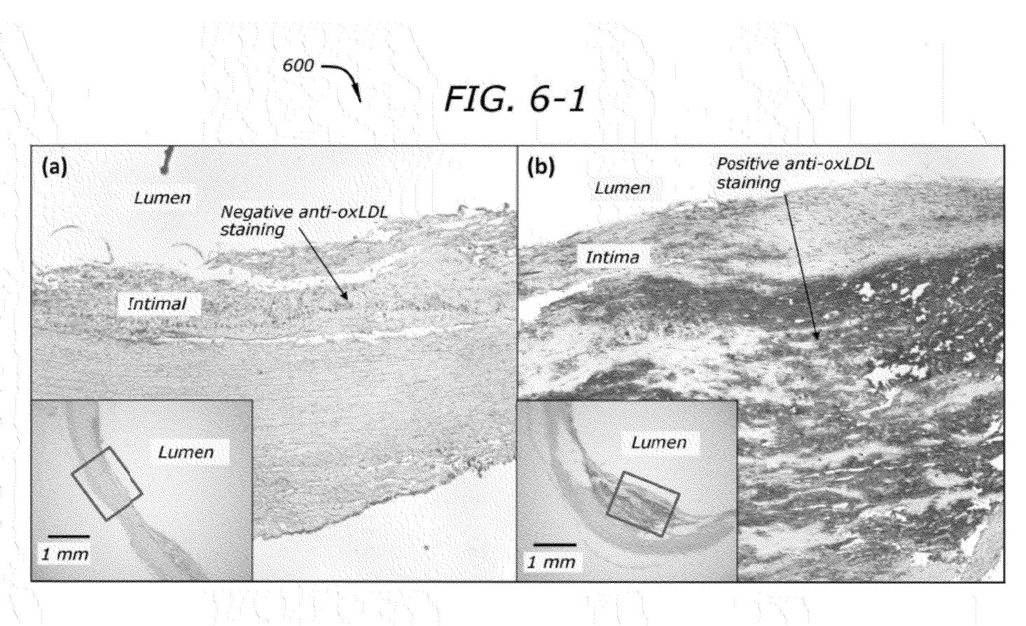

FIG. 5 depicts a set 500 of photographs of fatty streak tissue with accompanying endoluminal electrochemical impedance spectroscopic measurements according to the present disclosure. In FIG. 5, views (a)-(d) show endoluminal EIS measurements of fatty streaks. The fatty streak-absent site was stained negative for anti-oxLDL, as shown in FIG. 5a. Adjacent to this site was the fatty streak-rich site that was stained positive for both anti-oxLDL and Oil-Red-O, as shown in FIG. 5b. The EIS signals revealed the frequency-dependent differences between fatty streak-rich and fatty streak-free sites from 10 kHz to 300 kHz. The maximum difference in phase between the two measurements was at ˜10 kHz, as shown in FIG. 5c. The bar graph provided the statistically significant difference in impedance ranging from 10 kHz to 100 kHz (p<0.001, n=10 for fatty streak-rich, and n=8 for fatty streak-free endoluminal surface), as shown in FIG. 5d. Hence, a significant frequency-dependent increase in impedance in the fatty streaks (Stary Type II lesions) was demonstrated compared to lesion-free sites.

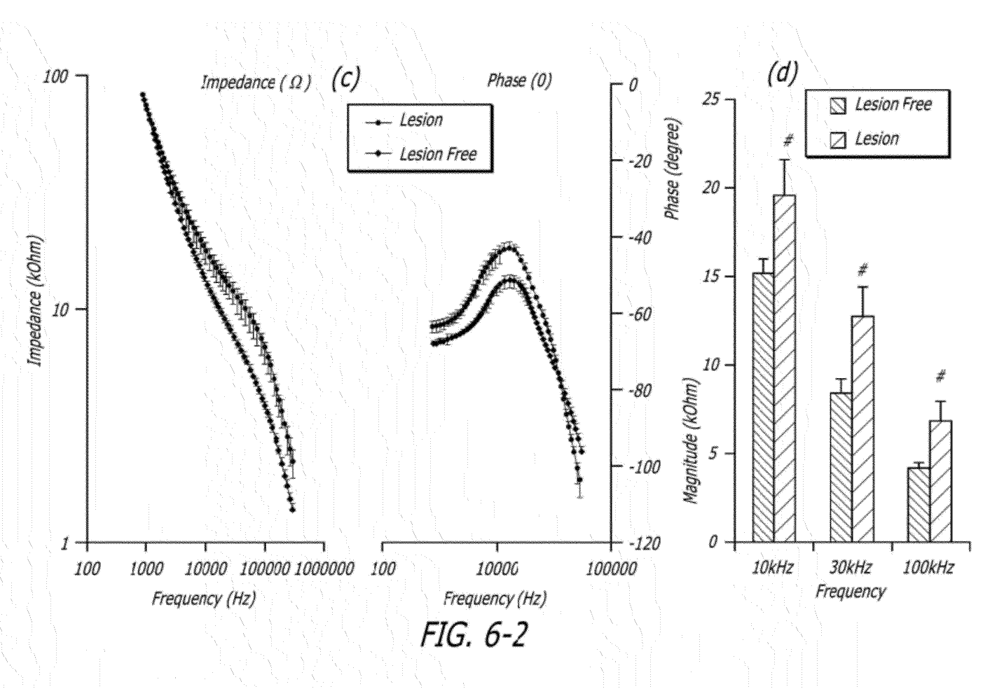

To address the inflammatory states underneath the fibrous caps, application of EIS was demonstrated in en face human carotid arteries. FIG. 6

FIG. 6 depicts a set 600 of photographs of oxLDL-rich fibrous atheroma tissue with accompanying endoluminal electrochemical impedance (EIS) spectroscopic measurements according to the present disclosure. Endoluminal EIS measurements were compared between intimal hyperplasia and early stage atheroma. Immunohistochemistry revealed a negative anti-oxLDL staining in region of intimal hyperplasia, as shown in FIG. 6a, but positive anti-oxLDL staining in the thin-capped (50 to 150 μm in cap thickness) atheroma that harbored a lipid core, as shown in FIG. 6b. Endoluminal EIS measurements revealed an increase in impedance from 10 kHz to 300 kHz in the thin-cap atheroma, as shown in FIG. 6c. The maximal phase differences between lesion-free and atheroma regions was also at 10 kHz. The bar graph further provided statistically significant differences in impedance ranging from 10 kHz to 100 kHz (p<0.01, n=6 for both thin-cap atheroma and lesion-free), as shown in FIG. 6d. Thus, a significant frequency dependent increase in impedance in the oxLDL-rich atheroma (Type III or IV) compared to the oxLDL-absent lesion-free regions is demonstrated.

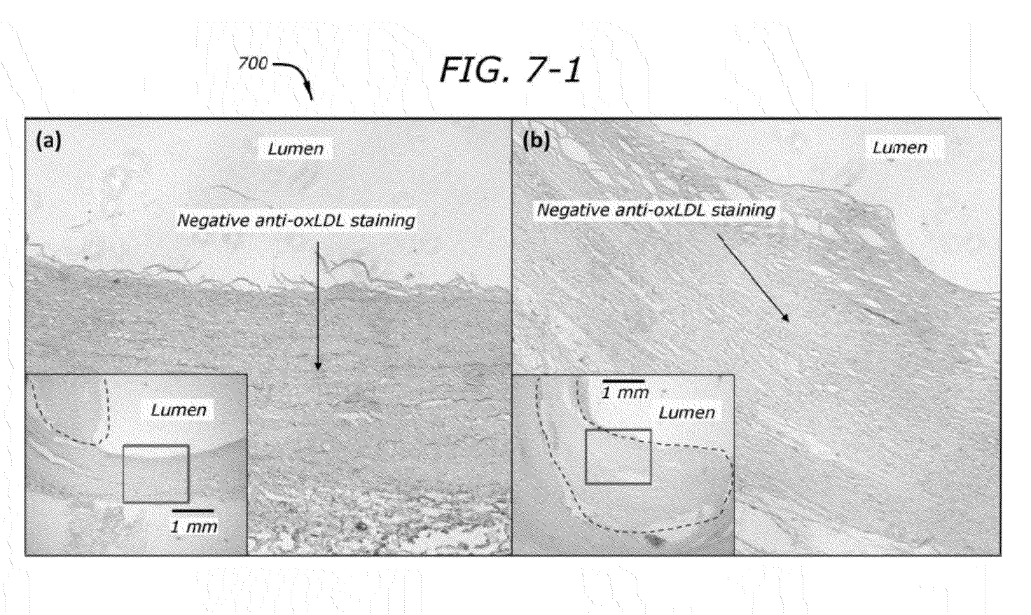

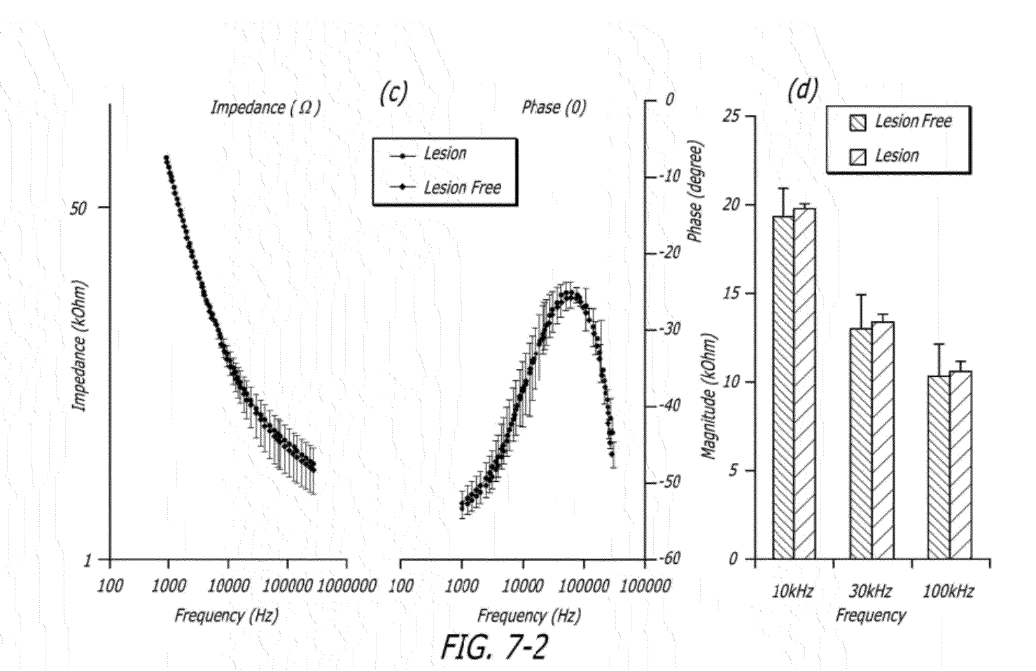

To assess the specificity for inflammatory states, frequency-dependent EIS measurements were performed to compare oxLDL-rich and -absent fibrous atheroma in en face human carotid arteries. FIG. 7 depicts a set 700 of photographs of oxLDL-absent fibrous atheroma tissue with accompanying endoluminal electrochemical impedance spectroscopic measurements according to the present disclosure. Endoluminal EIS measurements were compared between intimal hyperplasia and fibrous atheromas, followed by immunohistochemistry staining to reveal negative anti-oxLDL staining in both the lesion-free site, as shown in FIG. 7a, and the fibrous structure, as shown in FIG. 7b. Endoluminal EIS measurements showed a statistically insignificant difference in frequency-dependant impedance from 1 kHz to 300 kHz between the lesion-free sites and fibrous structures, as shown in FIG. 7c. The bar graph further supported statistically insignificant differences in impedances ranging from 10 kHz to 100 kHz (p>0.05, n=4 for lesion-free and n=6 for fibrous atheromas), as shown in FIG. 7d. Despite the presence of fibrous structure, insignificant changes in EIS measurements were consistent with the oxLDL-absent lesions (Type V).

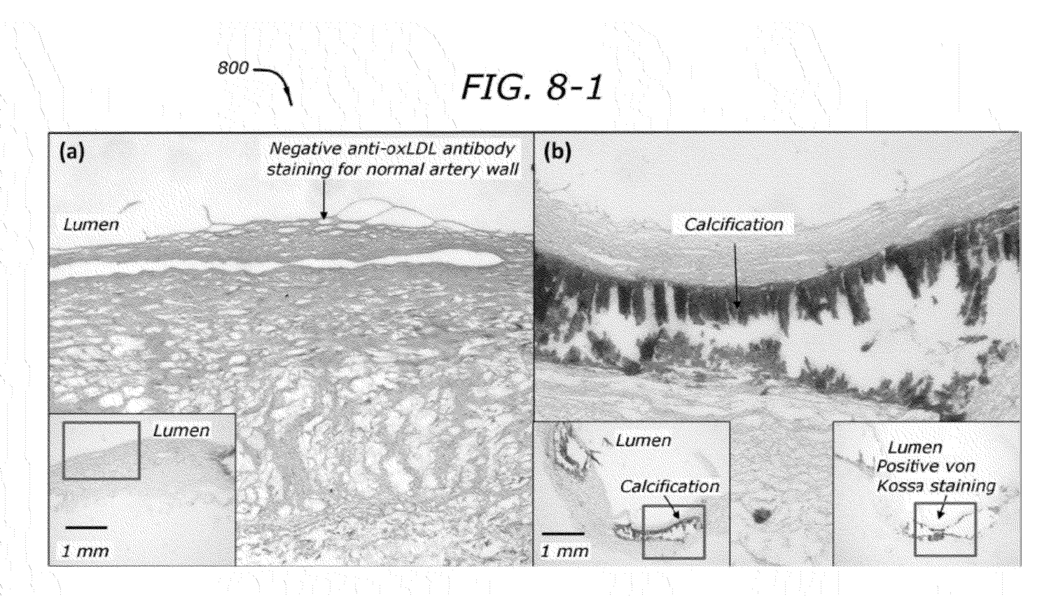

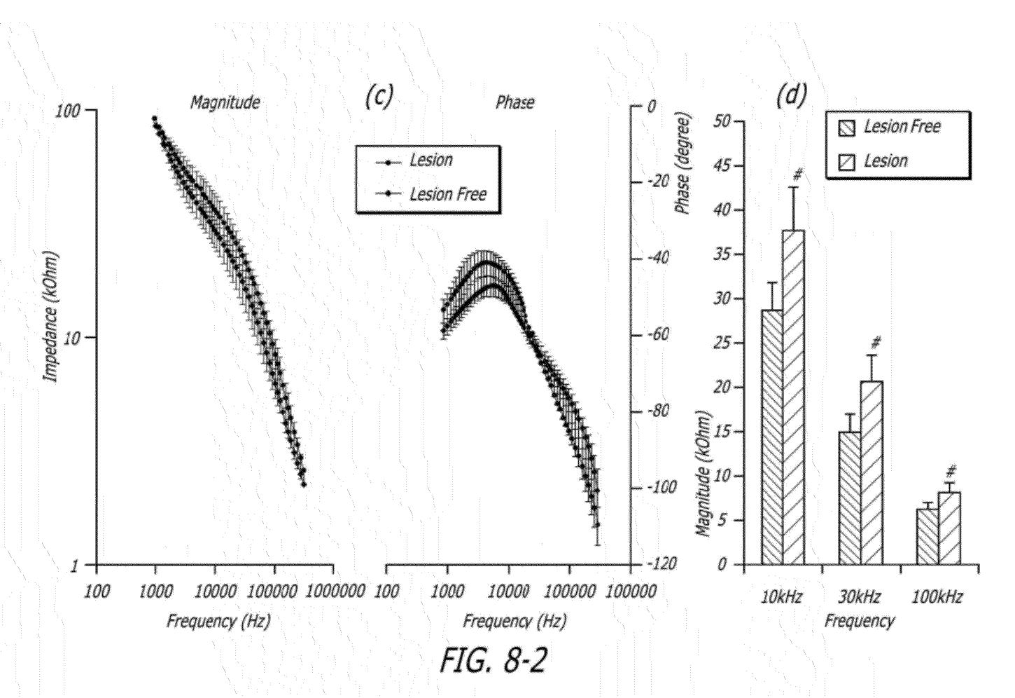

To further assess frequency-dependent EIS measurements in the presence of calcification, EIS measurements were performed in en face calcific atherosclerotic plaque from explants of human carotid arteries. FIG. 8 depicts a set 800 of photographs of calcification with accompanying endoluminal electrochemical impedance spectroscopic measurements according to the present disclosure. A hematoxylin-eosin (H&E) stain and immunohistochemistry revealed negative anti-oxLDL and von Kossa staining in the lesion-free sites, as shown in FIG. 8a, and positive von Kossa staining in the calcified lesions, as shown in FIG. 8b. The calcified core was dislodged during subsequent fixation, resulting in a void in the core. Endoluminal EIS measurements revealed an increase in impedance from 10 kHz to 100 kHz in the calcified lesions, as shown in FIG. 8c. The maximal phase difference between lesion-free and calcified regions was at ˜6 kHz. The bar graph demonstrated a statistically significant difference in impedance ranging from 10 kHz to 100 kHz (p<0.01, n=4 for calcification and n=6 for calcification-free regions), as shown in FIG. 8d. Therefore, it was demonstrated that calcific lesions (type VII) also engendered a significant increase in tissue impedance.

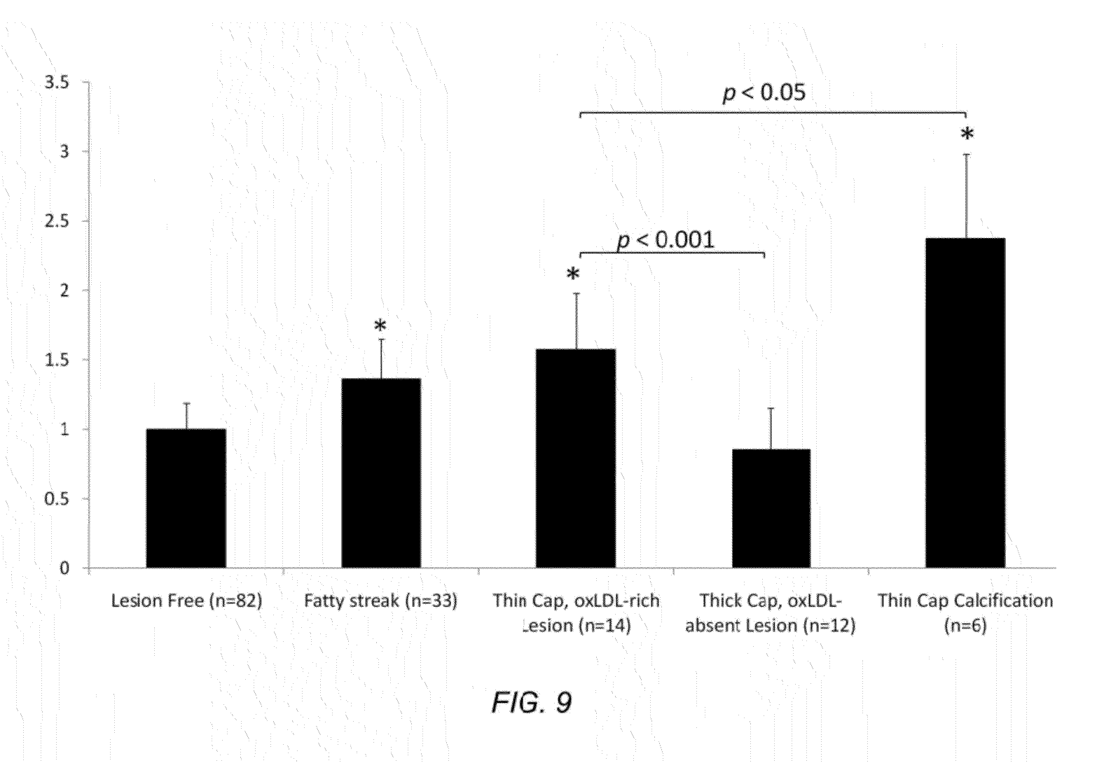

FIG. 9 depicts a set 900 of graphs showing the sensitivity and specificity of endoluminal electrochemical impedance spectroscopic measurements for an implemented embodiment of a concentric bipolar electrode sensor according to the present disclosure. To provide sensitivity and specificity of EIS signals, Rs (biological component resistance) values derived from the Equivalent Circuit 2, shown in FIG. 4b, were analyzed. Rs values from lesion sites were normalized to the mean Rs values from the lesion-free sites in the identical specimen. Statistically significant differences in Rs values were provided among Type II lesions (fatty streaks), Type III or IV lesions (thin-cap oxLDL-rich atheroma) and Type VII lesions (calcification) (*p<0.001) compared to the lesion-free sites. Statistically significant differences were corroborated between ox-LDL-rich and ox-LDL-absent fibrous atheromas (#p<0.001), and between oxLDL-rich and calcific lesions (p<0.05), as shown in FIG. 9. As a result, the electrochemical strategy of the implemented embodiments were seen as establishing sensitive and specific application of EIS measurements to detect inflammatory states of the atherosclerotic lesions.

FIG. 10 shows Table 1, which lists a classification scheme for atherosclerotic lesion stages, and Table 2, which shows a comparison of simulated circuit parameters for the equivalent circuits shown in FIG. 4.

Accordingly, systems, apparatus, and methods as described herein can provide concentric bipolar electrodes for electrochemical characterization of tissue structure and/or disease states such as fibrous atheromas and bioactive lipids in terms of impedance spectroscopy.

Aspects of the methods of EIS processing using concentric bipolar electrode assemblies outlined above may be embodied in programming. Program aspects of the technology may be thought of as “products” or “articles of manufacture” typically in the form of executable code and/or associated data that is carried on or embodied in a type of non-transitory machine readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer, processor, or device into another, for example, from a management server or host computer of the service provider into the computer platform of the application server that will perform the function of the push server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium may take many forms, including but not limited to, a tangible storage medium, a carrier wave medium or physical transmission medium. Non-volatile storage media include, for example, optical or magnetic disks, such as any of the storage devices in any computer(s), server(s), or the like, such as may be used to implement the push data service shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A system for electrochemical impedance spectroscopy, the system comprising:
a concentric bipolar electrode sensor assembly including an outer electrode and a center electrode disposed within the outer electrode, wherein the concentric bipolar electrode assembly is configured to supply an excitation voltage across the outer and center electrodes;
a memory configured to receive data from the concentric bipolar electrode assembly;
a processor connected to the memory; and
programming for execution by the processor, stored in the memory, wherein execution of the programming by the processor configures the system to perform functions, including functions to:
measure impedance across the outer and center electrodes.

2. The system of claim 1, wherein the functions further include correlating the measured impedance across the outer and center electrodes to a type of tissue.

3. The system of claim 1, further comprising a reference electrode.

4. The system of claim 3, wherein the concentric bipolar electrode assembly comprises a reference electrode comprising silver.

5. The system of claim 3, wherein the concentric bipolar electrode assembly comprises a reference electrode comprising silver-chloride.

6. The system of claim 1, wherein the center electrode comprises platinum.

7. The system of claim 1, wherein the outer electrode comprises platinum.

8. The system of claim 1, wherein the center electrode comprises gold.

9. The system of claim 1, wherein the outer electrode comprises gold.

10. The system of claim 1, wherein the concentric bipolar electrode assembly is configured to supply the excitation voltage with a desired frequency.

11. The system of claim 1, wherein the concentric bipolar electrode assembly is configured to supply the excitation voltage with a desired frequency range.

12. The system of claim 11, wherein the desired frequency range is from about 300 kHz to about 100 Hz.

13. The system of claim 1, wherein the concentric bipolar electrode assembly comprises a coaxial wire, wherein the center electrode is connected to a center signal wire of the coaxial wire, and wherein the outer electrode is connected to a ground wire of the coaxial wire.

14. The system of claim 11, wherein the center and outer electrodes are connected to the center signal wire and ground wire, respectively, by conductive epoxy.

15. The system of claim 13, further comprising biocompatible epoxy covering an exposed portion of the center signal wire and the ground wire.

16. The system of claim 13, wherein the concentric bipolar electrode sensor assembly comprises parylene separating the center and outer electrodes.

17. The system of claim 1, further comprising a potentiostat connected to the processor and configured to measure impedance.

18. An article of manufacture comprising:
a non-transitory machine-readable storage medium; and
executable program instructions embodied in the machine readable storage medium that when executed by a processor of a programmable computing device configures the programmable computing device to:
from electrical signals received from a bipolar concentric electrode sensor, measure impedance of biological tissue or material; and
provide an output signal indicative of the measure impedance to a display device.

19. The article of manufacture of claim 18, wherein the bipolar concentric electrode sensor comprises an outer electrode and a center electrode disposed within the outer electrode.

20. The article of manufacture of claim 19, wherein the executable program instructions further configure the programmable computing device to supply a voltage having a desired waveform across the outer and center electrodes, wherein the waveform includes a frequency range.

21. A method of assessing atherosclerotic plaques using electrochemical impedance spectroscopy, the method comprising:
providing to an arterial lumen, a concentric bipolar electrode sensor assembly including an outer electrode and a center electrode disposed within the outer electrode, wherein the concentric bipolar electrode assembly is configured to supply an excitation voltage across the outer and center electrodes, wherein the concentric bipolar electrode assembly comprises a coaxial wire, wherein the center electrode is connected to a center signal wire of the coaxial wire, and wherein the outer electrode is connected to a ground wire of the coaxial wire, and wherein the outer and center electrodes are configured on a surface of the sensor assembly that is configured for contact with the lumen;
applying a voltage across the center and outer electrodes;
measuring impedance across the center and outer electrodes; and
correlating the measured impedance to a type of biological tissue or material.

22. The method of claim 21, wherein correlating the measured impedance to a type of biological tissue or material comprises correlating the measured impedance to an atherosclerotic plaque.

23. The method of claim 22, wherein the atherosclerotic plaque is unstable.

24. The method of claim 23, wherein the atherosclerotic plaque is stable.

25. The method of claim 21, further comprising guiding the concentric bipolar electrode sensor assembly to a region of atherosclerotic plaque within the arterial lumen.
